(19) United States
(12) Patent Application Publication
(10) Pub. No.: US 2023/0364378 A1
(43) Pub. Date: Nov. 16, 2023

(54) REAL-TIME STRESS MANAGEMENT SYSTEM WITH STRESS DETECTION SENSOR AND BIOFEEDBACK LOOP ACTUATOR

(71) Applicant: E-Sentience, Inc., Newark, NJ (US)

(72) Inventor: Fernando Webb Vargas, Monterrey (MX)

(21) Appl. No.: 18/316,796

(22) Filed: May 12, 2023

Related U.S. Application Data
(60) Provisional application No. 63/341,217, filed on May 12, 2022.

(51) Int. Cl. A61M 21/02

(52) U.S. Cl. CPC A61M 21/02; A61M 2230/65; A61M 2230/20; A61M 2230/50; A61M 2205/3313; A61M 2205/3303; A61M 2021/0066

(57) ABSTRACT

Various implementations include a wearable stress-reducing system. The system includes one or more sensors, a stimulating device, and a control system. The sensors are configured to each measure at least one indicator of stress from a user. The stimulating device is configured to provide a stress-reducing stimulation to a portion of user's skin. The control system includes a controller that includes a logic processor that is configured to receive at least a stress indicator measurement from the sensors, compare the stress indicator measurement to a predetermined threshold, determine whether to modify the stress-reducing stimulation based on comparing the stress indicator measurement to the predetermined threshold, and, if a modification is needed, transmit an instruction to adjust the stress-reducing stimulation provided by the stimulating device. The stimulating device adjusts the stress-reducing stimulation enough to lower the at least one indicator of stress below the predetermined threshold.

### CROSS-REFERENCE TO RELATED APPLICATIONS

This application claims benefit of U.S. Provisional Application 63/341,217, filed on May 12, 2022, the content of which is hereby incorporated in its entirety.

### BACKGROUND

Seven of the major leading causes of death in the United States and other developed nations are related to stress: heart disease, cancer, stroke, injuries, suicide, chronic liver disease, and emphysema. Innovations that offer person-centered and cost-efficient means of stress management are essential for preventive healthcare. Moreover, technologies that contribute to stress reduction can improve the wellness and performance of people in the United States and globally.

Current commercially available wearable devices that define themselves as stress sensors measure stress through proxy variables, the most notable of which is heart rate variability (HRV), the variability of time between the beats of one's heart. There are several problems with this way of measuring bodily strain. First, the accuracy of HRV data collected by wrist or ring devices has been demonstrated to be inaccurate. HRV can only be accurately measured using a chest-based heart rate monitor, which is a device which most people are not willing to continuously wear. A similar issue arises for bulky wrist-based devices that aim to assess body strain levels over the prior 24 hours to give a readiness score in the morning. If users are unwilling or unable to comfortably wear these devices during periods of rest, the algorithms to calculate strain will fail to deliver useful information. In addition, these devices lack real-time actuation in response to the biometric data they are measuring. While a spike in body temperature or heart rate can indicate distress, these devices only provide feedback to the user based on a lagging conglomeration of data.

A direct measurement of an individual's stress is cortisol levels, which in practice is either quantified in a non-continuous matter through the use of high-performance liquid chromatography (HPLC), GC-MS (gas chromatography-mass spectrometry), or saliva and blood collection kits that must still be processed in a laboratory and rely on lab equipment to analyze. These tests tend to be inadequate given that cortisol levels change from hour-to-hour and day-to-day in response to external stimuli and dietary intake.

Previous patents like U.S. Pat. No. 6,833,274 B2 describe devices capable of measuring cortisol continually, however, like other commercial stress management devices, they lack a means to respond and reduce the stress levels they are measuring. Stress can be reliably measured through continuous monitoring of cortisol levels, but in order to provide effective stress reduction, a device must also be able to respond to the measured levels of cortisol.

Thus, there is a need for a device that can both accurately measure various biometric indicators of stress and also provide automated, instantaneous feedback to moderate such indicators as an effective means to measure and reduce users' stress levels.

Electrochemical sensors must meet several features to be able to measure the various chemical and physical signals of the body. These features include flexibility, high contact area, proper adhesion, and proper electrical contact. A special requirement for these sensors is a low impedance to ensure good transmission of electric energy and low polarization of any given electrode. Several patents address impedance, or conversely, increasing conductivity. In U.S. Pat. Nos. 4,895,169, 4,834,103, and 4,852,585, it is suggested to use an electrode element of tin and Stannous chloride directly upon a tin electrode to increase conductivity. In U.S. Pat. No. 4,352,359, an electrode is disclosed in which the impedance is lowered by the presence of a synthetic polymer containing at least five mole percent of monomer units containing a salt of a carboxylic acid. EP0836864 suggests using a conductive hydrogel adhesive containing an electrolyte distributed over the foil plate to increase the conductivity, potassium bromide being the preferred electrolyte. In U.S. Pat. No. 4,989,607, it is suggested to use a highly conductive hydrogel including a cohesive uniform mixture of polyvinyl pyrrolidone (PVP), a viscosity-enhancing hydrophilic polymer, and an electrolyte. All these methods work well to maximize conductivity, but they compromise the acquisition of measurements of individual molecular signals in the electrode medium.

U.S. Pat. App. Pub. No. 2021/0076988A1 describes sensors specifically devised to measure interstitial fluids in skin and other places. Using modern microfluidic electrodes, a dual anodic and cathodic compartment, and an electronic component, both continuous and quasi-continuous electrochemical measurements are disclosed. However, these electrodes have limited longevity, specifically with regards to precise measurements of analytes such as glucose, cortisol, dopamine, citric acid, sodium, and calcium ions, among others. Because of the electrodes' fixed lifespan, an end user of a chronic measurement device would need to periodically replace the electrodes or purchase a new device. Thus, there is a need for a device that includes electrodes that can take accurate, continuous chronic measurements for an extended amount of time.

### SUMMARY

In various implementation, a wearable stress-reducing system includes one or more sensors configured to each measure at least one indicator of stress from a user, a stimulating device configured to provide a stress-reducing stimulation to a portion of skin of the user, and a control system including a controller. The controller includes a logic processor that is configured to determine a stress indicator measurement based on the at least one indicator of stress measured by the one or more sensors, determine a modified stress-reducing stimulation when the stress indicator measurement is greater than a predetermined threshold, and control the stimulating device to provide the modified stress-reducing stimulation to lower the stress indicator measurement below the predetermined threshold.

In some implementations, the one or more sensors comprise a galvanic skin response (GSR) sensor.

In some implementations, the one or more sensors comprise an oxygenation sensor.

In some implementations, the one or more sensors comprise a temperature sensor. In some implementations, the temperature sensor comprises an infrared (IR) temperature sensor.

In some implementations, the one or more sensors are configured to each measure a same at least one indicator of stress from a fluid from the user. In some implementations, the fluid is sweat.

In some implementations, the one or more sensors comprise an electrochemical sensor.

In some implementations, the at least one indicator of stress comprises cortisol.

In some implementations, the stress-reducing stimulation comprises cooling.

In some implementations, the stress-reducing stimulation comprises warming.

In some implementations, the stress-reducing stimulation comprises mechanical stimulation.

In some implementations, the one or more sensors, the stimulating device, and the control system are included in a single, wearable device. In some implementations, the wearable device is configured to be worn on an upper arm of the user. In some implementations, the wearable device is configured to be worn behind an ear of the user.

In some implementations, the one or more sensors comprise two or more sensors, wherein the two or more sensors comprise a same type of sensor.

In some implementations, the wearable stress-reducing system further includes a degradable layer disposed adjacent at least one of the two or more sensors such that the degradable layer is configured to be disposed between at least one of the two or more sensors and the user. In some implementations, the two or more sensors comprise three or more sensors, the degradable layer being disposed adjacent at least a first of the three or more sensors and a second of the three or more sensors, wherein the degradable layer has a thickness as measured in a direction from the sensor toward the user, wherein the thickness of the degradable layer adjacent the first of the three or more sensors is greater than the thickness of the degradable layer adjacent the second of the three or more sensors. In some implementations, the degradable layer is degradable by heat. In some implementations, the degradable layer is not disposed adjacent an active sensor of the two or more sensors such that the degradable layer is configured not to be disposed between the active sensor and the user, wherein the logic processor is further configured to determine whether the active sensor exceeds a sensor degradation threshold, and if the active sensor exceeds the sensor degradation threshold, transmit an overpotential instruction to an inactive sensor of the two or more sensors to cause the inactive sensor to produce enough heat to cause degradation of the degradable layer adjacent the inactive sensor.

In other implementations, a wearable sensor array system includes two or more sensors, configured to each measure at least one measurement of a user, and a degradable layer disposed adjacent at least one of the two or more sensors such that the degradable layer is configured to be disposed between at least one of the two or more sensors and the user.

In some implementations, the two or more sensors comprise three or more sensors, the degradable layer being disposed adjacent at least a first of the three or more sensors and a second of the three or more sensors, wherein the degradable layer has a thickness as measured in a direction from the sensor toward the user, wherein the thickness of the degradable layer adjacent the first of the three or more sensors is greater than the thickness of the degradable layer adjacent the second of the three or more sensors.

In some implementations, the degradable layer is degradable by heat. In some implementations, the degradable layer is not disposed adjacent an active sensor of the two or more sensors such that the degradable layer is configured not to be disposed between the active sensor and the user. In some implementations, the system further includes a control system comprising a controller, the controller comprising a logic processor that is configured to determine whether the active sensor exceeds a sensor degradation threshold, and if the active sensor exceeds the sensor degradation threshold, transmit an overpotential instruction to an inactive sensor of the two or more sensors to cause the inactive sensor to produce enough heat to cause degradation of the degradable layer adjacent the inactive sensor.

In some implementations, the two or more sensors comprise a galvanic skin response (GSR) sensor.

In some implementations, the two or more sensors comprise an oxygenation sensor.

In some implementations, the two or more sensors comprise a temperature sensor. In some implementations, the temperature sensor comprises an infrared (IR) temperature sensor.

In some implementations, the two or more sensors are configured to each measure at least one indicator of stress from the user. In some implementations, the two or more sensors are configured to each measure the same at least one indicator of stress from a fluid from the user. In some implementations, the fluid is sweat. In some implementations, the at least one indicator of stress comprises cortisol.

In some implementations, the two or more sensors comprise an electrochemical sensor.

In some implementations, the two or more sensors and the degradable layer are included in a single, wearable device. In some implementations, the wearable device is configured to be worn on the upper arm of the user. In some implementations, the wearable device is configured to be worn behind the ear of the user.

In some implementations, the two or more sensors comprise a same type of sensor.

In some implementations, the sensor degradation threshold is based on an amount of time that the active sensor has been activated.

In some implementations, the sensor degradation threshold is based on a deviation from an expected measurement of the active sensor.

In some implementations, the two or more sensors comprise twelve or more sensors.

This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements.

### BRIEF DESCRIPTION OF THE DRAWINGS

Example features and implementations are disclosed in the accompanying drawings. However, the present disclosure is not limited to the precise arrangements and instrumentalities shown.

FIG. 1A shows a perspective view of a wearable stress-reducing system, according to one implementation.

FIG. 1B shows a perspective view of the wearable stress-reducing system of FIG. 1A worn by a user.

FIGS. 2A-2D show schematic views of sensor configurations.

FIG. 3 shows a side view of the degradable layer covering the sensors of the wearable stress-reducing system of FIG. 1A.

### DETAILED DESCRIPTION

The devices, systems, and methods disclosed herein provide for a novel wear-and-forget “second skin” that enables users to measure stress levels directly, reliably, and accurately in real-time while providing an immediate stress-reducing actuation based on a biofeedback loop that includes a cooling sensation.

Disclosed herein is a stress monitoring, managing, and mitigating device utilizing an array of optical, electrical, and electrochemical sensors to gather biological signals for stress which establishes a baseline level of stress and introduces a cooling response proportional to the amount of stress above the baseline. The system can both gather data and learn from the user to provide the user and trusted partners insight into their physical and mental wellbeing. The sensors gather data, which in turn, is processed by the controller and sent to the appropriate software for terminal visualization and used to decide an appropriate cooling response.

The main improvements of the devices disclosed herein reside in the reliability of the stress level measurement and the implementation of a cooling system that provides stress relieving effects when normal stress levels are exceeded. There is currently no commercially available device that continuously measures cortisol levels or levels of other hormones. A few existing devices evaluate users' stress or strain levels through indirect measures of stress such as heart rate, heart rate variability, and temperature. Unlike other wearables marketed for stress response, the wearable devices disclosed herein work in-the-moment to provide immediate feedback about stress levels and produce a stress-reducing cooling sensation. Continuously measuring cortisol levels provides ways to quantify stress levels accurately in real-time.

By using degradable polymer coatings, an array of electrodes can be used to extend the lifetime of a sensor electrode. Working electrodes in an electrochemical cell are subject to degradation over their lifetime, both through natural and electrochemical means. This phenomenon limits their ability to be used accurately over long periods of time. This happens with organic, inorganic, and biological electrochemical sensors. For electrodes designed to take chronic, continuous measurements, this is a serious issue. To address this problem the devices disclosed herein include an array of electrodes, where only one is active at the time. The other electrodes are covered in a protective, thermically degradable layer. When the active electrode starts to show signs of degradation, the next electrode warms up gently to shed the protective layer. Once this process is complete, the active status changes from one electrode to the next.

Electrodes have a fixed durability, specifically with regards to precise measurements of analytes such as, but not limited to, glucose, cortisol, dopamine, citric acid, sodium, and calcium ions among others. A device that requires the user to constantly replace electrodes is not a commercially feasible means to continuously measure such analytes over a long period of time. Constantly replacing electrodes is not necessarily the best approach for chronic measurement.

Herein, are disclosed methods to achieve chronic, continuous measurement by introducing a series of protected electrodes instead of one. Henceforth, the electrode currently being used shall be known as the “active electrode” or “active sensor” while the others shall be discussed as the “reserve electrodes” or “reserve sensors.” In each array, the first electrode is numbered one and given the status of active, while the reserve electrodes are numbered into the Nth number, which is the last electrode on the array. Each electrode has a predetermined time or a logical condition for the active electrode to turn off and pass active status to the next numbered reserve electrode. This process repeats until all the electrodes in reserve have been utilized. The protective (or sacrificial) layer on the reserve electrode is made of a thermal degradable biocompatible polymer layer with a thickness that can vary from a nanometer to 10 microns depending on the desired size and projected longevity of the electrode array. The higher the reserve number is, the thicker the layer it has, to isolate the active components of the electrode from degradation further. Depending on the type of electrode underneath the degradable protective layer, an overpotential can be given to heat up the electrode after the electrode has been activated to further shed the remains of the protective layer. Through this method, it is possible to prolong the durability of electrodes that would otherwise be too labile to provide continuous measurements for weeks or months. Arrays do not have limited sizes or structures and are programmable and customizable to ensure reproducible, precise, and accurate electrochemical measurements of analytes in any type of interstitial fluid.

Various implementations include a wearable stress-reducing system. The system includes one or more sensors, a stimulating device, and a control system. The one or more sensors are configured to each measure at least one indicator of stress from a user. The stimulating device is configured to provide a stress-reducing stimulation to a portion of skin of the user. The control system includes a controller, and the controller includes a logic processor that is configured to receive at least a stress indicator measurement from the one or more sensors, compare the stress indicator measurement to a predetermined threshold, determine whether to modify the stress-reducing stimulation based on comparing the stress indicator measurement to the predetermined threshold, and if a modification is needed, transmit an instruction to adjust the stress-reducing stimulation provided by the stimulating device. The stimulating device adjusts the stress-reducing stimulation enough to lower the at least one indicator of stress below the predetermined threshold.

Various other implementations include a wearable sensor array system. The system includes two or more sensors and a degradable layer. The two or more sensors are configured to each measure at least one measurement of a user. The degradable layer is disposed adjacent at least one of the two or more sensors such that the degradable layer is configured to be disposed between the at least one of the two or more sensors and the user.

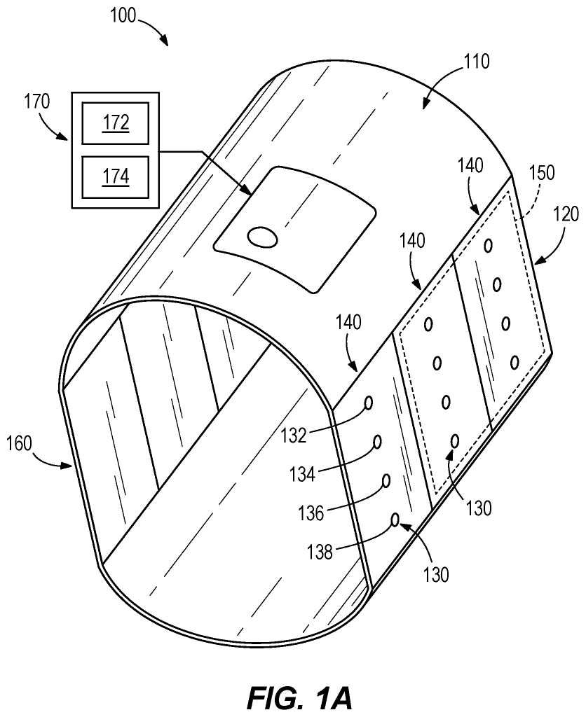

FIGS. 1A and 1B show a wearable stress-reducing system 100, according to various aspects of some implementations. The system 100 includes a band 110, a sensor portion 120, a stimulating device 160, and a control system 170.

The band 110 is made of a woven elastic material, but in other implementations, the band can be made of any other material suitable for being worn by the user. The sensor portion 120, stimulating device 160, and control system 170 are coupled to the resilient band 110 such that the sensor portion 120 and the stimulation device 160 are in contact with the skin of a user 190 when worn by the user 190. The band 110 of the wearable system 100 shown in FIGS. 1A and 1B is configured to be worn on the upper arm of the user 190, but in other implementations, the band can be configured to be work on any portion of the user's body. In some implementations, the device does not include a band and instead includes an adhesive or other coupling means for coupling the device to a portion of the body of a user. In some implementations, the device is configured to couple to a portion of the body of a user such that the sensor portion can measure an indicator of stress within one or more fluids in or on the user's body (e.g., sweat, blood, urine, saliva, interstitial fluid, mucus, hair oil). In some implementations, the wearable device is configured to be worn behind the ear of the user, the back of the neck of the user, in the hair of the user, or along the pelvic region of the user. Although the system 100 shown in FIGS. 1A and 1B includes a band 110 to couple the arm to a user 190, in other implementations, the system can include an adhesive patch, a clip, or a garment. As used herein, the term “user” is defined as any animal, including humans.

The sensor portion 120 includes an array of twelve sensors 130 arranged in three rows 140 of four sensors 130. Each of the four sensors 130 in a given row 140 are configured to each measure a different indicator of stress of a user 190. Each row 140 includes the same four sensors 130, and thus, the sensor portion 120 of the system 100 includes three of each type of sensor 130.

Each row 140 includes a first sensor 132, a second sensor 134, a third sensor 136, and a fourth sensor 138. The first sensor 132 is an infrared (IR) temperature sensor. The IR temperature sensor is configured to measure the temperature of the user's skin and to measure the user's heart rate. The second sensor 134 is an oxygen sensor. The oxygen sensor is used to measure the user's pulse. The third sensor 136 is a galvanic skin response (GSR) sensor. The GSR sensor is configured to measure the tenseness of the user's muscles. The fourth sensor 138 is an electrochemical sensor configured to measure cortisol in the user's sweat. As used herein, the term “electromechanical sensor” refers to any sensor that transforms mechanical stimulus into electrical signals.

Each of these measurements (i.e., temperature, heart rate, pulse, muscle tenseness, cortisol levels) can be an indicator of stress for the user 190. Alone, each of these measurements can be triggered by any number of causes other than stress. However, by monitoring multiple indicators of stress together, causes for the changes to these measurements other than stress can be ruled out, thus providing a more accurate measurement of a user's stress levels.

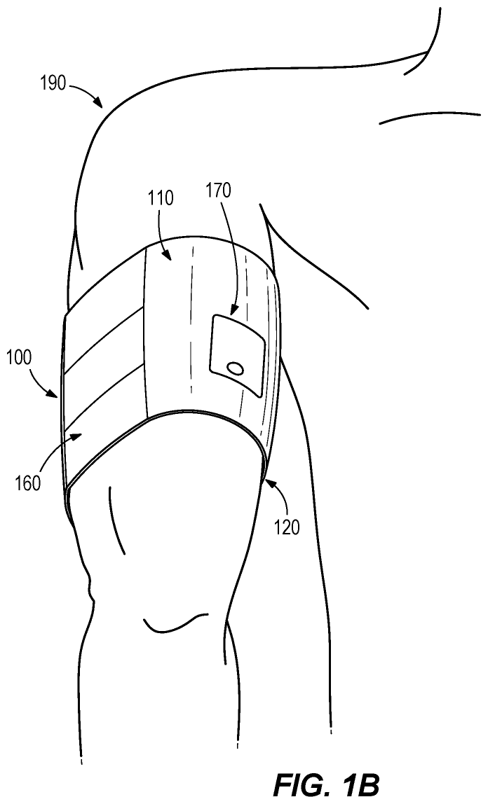

Although the sensor portion 120 includes twelve total sensors 130 arranged in three rows 140 of the same four sensors 130, in other implementations, the sensor portion can include any number of the same one or more sensors. In some implementations, the sensor portion includes an array of sensors in any configuration. For example, FIGS. 2A-2D show various different configurations of sensors 130 other than rows.

Although the electromechanical sensor 130 of the sensor portion 120 shown in FIGS. 1A and 1B is configured to measure cortisol, in other implementations, the sensor can be configured to measure one or more biomarkers that are indicators of stress in a bodily fluid of the user, including, but not limited to glucose, dopamine, citric acid, sodium, calcium ions, or lactic acid. The bodily fluid in which the indicator of stress is measured by the electromechanical sensor 130 in FIGS. 1A and 1B is sweat, however in other implementations, the bodily fluid can be any other bodily fluid, such as blood, urine, saliva, interstitial fluid, mucus, or hair oil.

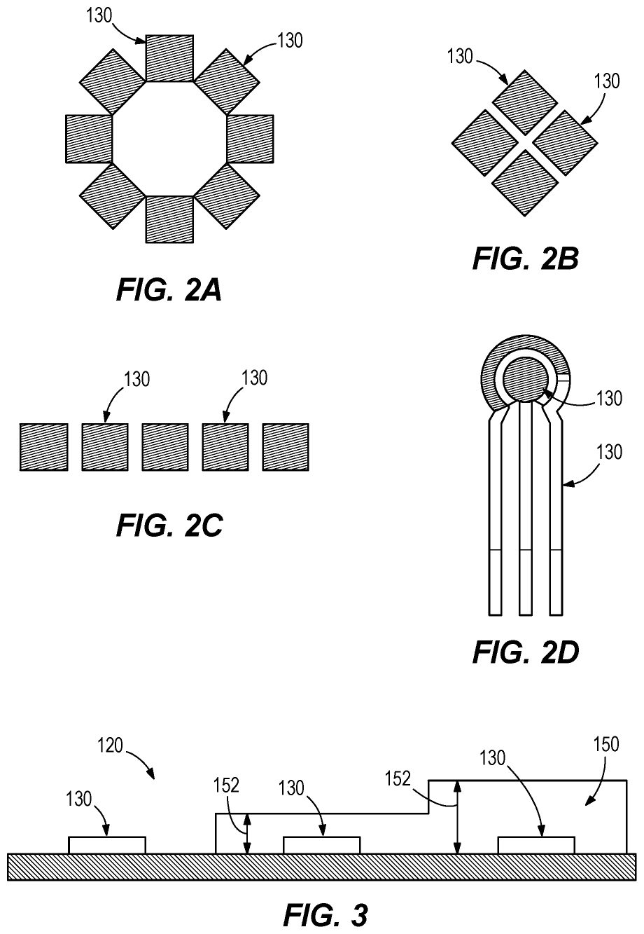

The sensor portion 120 further includes a degradable layer 150, as shown in FIG. 3. The degradable layer 150 is disposed over two of the three rows 140 of sensors 130 such that the degradable layer 150 is disposed between the sensors 130 and the skin of the user 190 when the system 100 is worn. The degradable layer 150 is degradable over time by the heat of the environment, the user 190, and the electronics of the system 100.

As shown in FIG. 3, the degradable layer 150 has a stepped, tapering level of thickness 152 as measured in a direction from the sensor 130 toward the user 190. The portion of the degradable layer 150 that covers the row 140 of sensors 130 adjacent the uncovered row 140 has the thinnest thickness 152 and the thickness 152 of the degradable layer 150 is thicker with each row 140 that is further away from the uncovered row 140.

As mentioned above, the sensors 130 included in the sensor portion 120 have a useful life duration, after which the sensors 130 no longer function as intended (e.g., no longer measure accurately or no longer function at all). This poses an issue for the life span of any product that uses such sensors 130. However, by including an array of sensors 130 in the sensor portion 120 and only having one row 140 of active sensors 130 uncovered at a time provides the system 100 with the ability to automatically exchange the sensors 130 once the sensors 130 are no longer useable and extends the useful lifetime of the sensor substantially.

The degradable layer 150 of the system 100 shown in FIGS. 1A and 1B is degradable at least partially by the heat of the body of the user 190 wearing the system 100. Thus, as the sensors 130 of the uncovered row 140 of active sensors 130 are used, the degradable layer 150 begins to degrade. The thicknesses 152 of the tapered degradable layer 150 is predetermined to degrade such that the portion of the degradable layer 150 covering the row 140 of reserve sensors 130 adjacent the uncovered row 140 of active sensors 130 will fully degrade by the time the uncovered row 140 of active sensors 130 become unusable. The system 100 is then programed to stop using the originally uncovered row 140 of active sensors 130 and begin measuring stress indicators using the newly uncovered adjacent row 140 of reserved sensors 130. At the same time, the tapered degradable layer 150 covering the remaining row 140 of reserved sensors 130 will have partially degraded such that the currently covered third row 140 of reserved sensors 130 will be uncovered by the time the second row 140 of sensors 130 have become unusable. For implementations in which there are more than three rows of sensors, this process will continue until the entire degradable layer has fully degraded such that all rows of reserved sensors have been uncovered. Once the final row 140 of reserved sensors 130 has become unusable, the user 190 can replace the entire sensor portion 120 of the system 100 with a new sensor portion 120 having a new array of sensors 130 partially covered by a new degradable layer 150.

Although the system 100 shown in FIGS. 1A and 1B includes a tapered degradable layer 150 covering two of the three rows 140 of sensors 130, in other implementations, the degradable layer can be configured and tapered such that the degradable layer will degrade in any predetermined way to uncover any number of sensors in any order. This is especially true of other implementations of sensor 130 configurations, such as those disclosed in FIGS. 2A-2D, to allow for the degradable layer to degrade in the way intended and described herein.

The stimulating device 160 is coupled to the band 110 and includes a thermoelectric module configured to provide cooling to a portion of the skin of the user 190 as a stress-reducing stimulation. However, in some implementations, the stimulating device can alternatively or additionally include warming, mechanical stimulation (e.g., tapping, vibration, rubbing, rolling), and/or electrical stimulation. The cooling and/or warming can be created in any known way, such as by thermoelectric modules, fluid heat exchange, refrigeration, electrical resistance, or any combination thereof.

The control system 170 includes a controller having a logic processor 172 that is configured to perform a series of stress-reducing actions based on received measurements and computer readable instructions 174. The computer readable instructions 174 cause the processor 172 to receive at least one stress indicator measurement from one or more of the sensors 130 in the uncovered row 140 of active sensors 130.

The computer readable instructions 174 then cause the processor 172 to compare the stress indicator measurement to a predetermined threshold. The predetermined threshold can be a set value and/or a baseline determined based on the stress indicator measurements over time.

The computer readable instructions 174 then cause the processor 172 to determine whether to modify the stress-reducing stimulation based on comparing at least one received stress indicator measurement to the predetermined threshold. This determination can be made based on a deviation from the predetermined threshold.

If the processor 172 determines that a modification is needed, the computer readable instructions 174 then cause the processor 172 to transmit an instruction (e.g., a modified stress-reducing stimulation) to adjust the stress-reducing stimulation provided by the stimulating device 160. The adjustment can be activating/deactivating one or more stress-reducing stimulations, increasing or decreasing the intensity of one or more stress-reducing stimulations, pulsing or alternating one or more stress-reducing stimulations, or any combination thereof. In all cases, the stimulating device 160 adjusts the stress-reducing stimulation enough to lower the at least one indicator of stress below the predetermined threshold.

In some implementations, the wearable stress-reducing system 100 includes one or more sensors 130. The one or more sensors are configured to each measure at least one indicator of stress from the user 190. The stimulating device 160 is configured to provide a stress-reducing stimulation to a portion of skin of the user 190. The control system 170 includes a controller including the logic processor 172 and is configured to determine the stress indicator measurement based on the at least one indicator of stress measured by the one or more sensors 130, determine a modified stress-reducing stimulation when the stress indicator measurement is greater than a predetermined threshold, and control the stimulating device 160 to provide the modified stress-reducing stimulation to lower the stress indicator measurement below the predetermined threshold.

The controller 170 can also be used to cause the acceleration of the degradation of the degradable layer 150 if needed. The computer readable instructions 174 can cause the processor 172 to determine whether the active sensor 130 exceeds a sensor degradation threshold. The sensor degradation threshold can be based on a continued deviation from an expected measurement of the active sensor 130 or based on an amount of time that the active sensor 130 has been activated. If the active sensor 130 exceeds the sensor degradation threshold, the computer readable instructions 174 cause the processor 172 to transmit an overpotential instruction to one or more sensors 130 in a row 140 of reserved sensors 130 (i.e., inactive sensors) that are covered by the degradable layer 150. The overpotential instruction causes the one or more sensors 130 (i.e., inactive sensors) to produce excess heat enough to cause accelerated degradation of the degradable layer 150 adjacent the one or more reserved sensors 130. The controller 170 can then discontinue the use of the row 140 of active sensors 130 and begin to use the newly uncovered row 140 of reserved sensors 130.

Although the sensors 130, the stimulating device 160, and the control system 170 shown in FIGS. 1A and 1B are included in a single, wearable device, in other implementations, one or more of the sensors, the stimulating device, or the control system could be included in a separate wearable device.

A number of example implementations are provided herein. However, it is understood that various modifications can be made without departing from the spirit and scope of the disclosure herein. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. Although the terms “comprising” and “including” have been used herein to describe various implementations, the terms “consisting essentially of” and “consisting of” can be used in place of “comprising” and “including” to provide for more specific implementations and are also disclosed.

Disclosed are materials, systems, devices, methods, compositions, and components that can be used for, can be used in conjunction with, can be used in preparation for, or are products of the disclosed methods, systems, and devices. These and other components are disclosed herein, and it is understood that when combinations, subsets, interactions, groups, etc. of these components are disclosed that while specific reference of each various individual and collective combinations and permutations of these components may not be explicitly disclosed, each is specifically contemplated and described herein. For example, if a device is disclosed and discussed each and every combination and permutation of the device are disclosed herein, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed systems or devices. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

1. A wearable stress-reducing system, the system comprising:
one or more sensors configured to each measure at least one indicator of stress from a user;
a stimulating device configured to provide a stress-reducing stimulation to a portion of skin of the user; and
a control system comprising a controller, the controller comprising a logic processor that is configured to:
determine a stress indicator measurement based on the at least one indicator of stress measured by the one or more sensors,
determine a modified stress-reducing stimulation when the stress indicator measurement is greater than a predetermined threshold, and
control the stimulating device to provide the modified stress-reducing stimulation to lower the stress indicator measurement below the predetermined threshold.

2. The system of claim 1, wherein the one or more sensors comprise a galvanic skin response (GSR) sensor.

3. The system of claim 1, wherein the one or more sensors comprise an oxygenation sensor.

4. The system of claim 1, wherein the one or more sensors comprise a temperature sensor.

5. The system of claim 4, wherein the temperature sensor comprises an infrared (IR) temperature sensor.

6. The system of claim 1, wherein the one or more sensors are configured to each measure a same at least one indicator of stress from a fluid from the user.

7. The system of claim 6, wherein the fluid is sweat.

8. The system of claim 1, wherein the one or more sensors comprise an electrochemical sensor.

9. The system of claim 1, wherein the at least one indicator of stress comprises cortisol.

10. The system of claim 1, wherein the stress-reducing stimulation comprises cooling.

11. The system of claim 1, wherein the stress-reducing stimulation comprises warming.

12. The system of claim 1, wherein the stress-reducing stimulation comprises mechanical stimulation.

13. The system of claim 1, wherein the one or more sensors, the stimulating device, and the control system are included in a single, wearable device.

14. The system of claim 13, wherein the wearable device is configured to be worn on an upper arm of the user.

15. The system of claim 13, wherein the wearable device is configured to be worn behind an ear of the user.

16. The system of claim 1, wherein the one or more sensors comprise two or more sensors, wherein the two or more sensors comprise a same type of sensor.

17. The system of claim 16, further comprising a degradable layer disposed adjacent at least one of the two or more sensors such that the degradable layer is configured to be disposed between at least one of the two or more sensors and the user.

18. The system of claim 17, wherein the two or more sensors comprise three or more sensors, the degradable layer being disposed adjacent at least a first of the three or more sensors and a second of the three or more sensors, wherein the degradable layer has a thickness as measured in a direction from the sensor toward the user, wherein the thickness of the degradable layer adjacent the first of the three or more sensors is greater than the thickness of the degradable layer adjacent the second of the three or more sensors.

19. The system of claim 17, wherein the degradable layer is degradable by heat.

20. The system of claim 19, wherein the degradable layer is not disposed adjacent an active sensor of the two or more sensors such that the degradable layer is configured not to be disposed between the active sensor and the user,
wherein the logic processor is further configured to:
determine whether the active sensor exceeds a sensor degradation threshold; and
if the active sensor exceeds the sensor degradation threshold, transmit an overpotential instruction to an inactive sensor of the two or more sensors to cause the inactive sensor to produce enough heat to cause degradation of the degradable layer adjacent the inactive sensor.

21-40. (canceled)
